(19) United States
(12) Patent Application Publication
(10) Pub. No.: US 2003/0175698 A1
(43) Pub. Date: Sep. 18, 2003

(54) Retrovirus-specific oligonucleotides and a method for measuring retroviral titer using same

(76) Inventors: Jong-Mook Kim, Seoul (KR); Hongchan Cho, Seoul (KR); Soomin Shim, Seoul (KR); Sunyoung Kim, Seoul (KR)

Correspondence Address: David A Einhorn, Anderson Kill & Olick, 1251 Avenue of the Americas, New York, NY, 10020, US

(21) Appl. No.: 10/332,679

(22) PCT Filed: May 9, 2002
(86) PCT No.: PCT/KR02/00858

(30) Foreign Application Priority Data
May 10, 2001 (KR) 2001/25476

(51) Int. Cl. C12Q 1/70; C12Q 1/68; C12N 15/86

(52) U.S. Cl. 435/5; 435/6; 435/456

(57) ABSTRACT

The present invention provides retrovirus-specific oligonucleotides and a method for measuring retroviral titer using same as primers in polymerase chain reaction (PCR). As the inventive method makes it possible to rapidly quantify measure retroviral DNA inserted into the genome of a target cell or the presence of retroviral RNA in a solution, it can be effectively used in the measurement of retroviral vector for gene therapy, the detection of replication competent retrovirus (RCR) in the sera of a subject receiving gene therapy, or the diagnosis of wild-type retrovirus.

### FIELD OF THE INVENTION

The present invention relates to retrovirus-specific oligonucleotides and a method for measuring retroviral titer using same as primers for polymerase chain reaction (PCR).

### BACKGROUND OF THE INVENTION

Retrovirus vector is the most frequently used gene-delivery vehicle among various types of vectors for gene therapy which is employed in more than 40% of approved clinical trials (Yu et al, Gene Therapy 7:797-804, 2000). The measurement of retroviral titer is a matter of critical importance in the use of retrovirus vector for gene therapy, for a retroviral titer of over a certain level (10⁵˜10⁶ cells/ml) is required in gene therapy. If the viral titer does not reach that level, the gene delivery efficiency becomes so low that it is not possible to achieve the intended cure. Therefore, it is necessary to measure the viral titer in evaluating a retrovirus-producing cell line for gene therapy or comparing different kinds of retrovirus vectors.

Further, a method for measuring retroviral titer can be advantageously used for detecting replication-competent retrovirus (RCR) which may be present as a contaminant in retrovirus used for gene therapy. When contaminated by RCR, virus can be produced in target cells besides in packaging cell lines, and thus, it is very important to quantify the retroviral titer and examine the presence of RCR.

Several methods have been developed for measuring the retroviral titer, which can be divided into three groups as follows.

The first is a method for measuring the retroviral titer in the supernatant of a cell culture solution by determining the amount of viral genomic RNA using reverse transcriptase activity, dot blot hybridization reaction, or semi-quantitative RT-PCR. However, this method measures not the number of virus capable of delivering a therapeutic gene to a target cell but the number of total virus including defective virus, and therefore, it has the problem of unsatisfactorily low precision and accuracy.

The second method is to indirectly measure the expression level of a gene introduced in the target cell by retrovirus. It uses various marker genes to discriminate the cells with introduced gene from others. This method measures viral titer by counting the number of colonies which are selectively cultured in a culture solution containing a drug, or by determining the number of infected cells through selective staining of target cells. Examples of the marker gene which may be employed in this method are neomycin phosphotransferase (Neo), hygromycin B phosphotransferase (Hygro), β-galactosidase (LacZ) and β-glucuronidase (GUS). However, since it has been reported that the products of such marker genes may trigger immune responses in animal model or clinical studies, the use of a retrovirus vector having only a therapeutic gene without a marker gene has become preponderant Therefore, the second method cannot be used in measuring the titer of retrovirus having only a therapeutic gene.

The third method is to determine retroviral titer by measuring the amount of retrovirus genomic DNA introduced into target cell genome. For this purpose, southern blot analysis is widely used. Since this method uses a selective probe for hybridization, it can be applied to measuring retroviral titer regardless of the introduced gene. However, southern blot analysis is not a quantitative tool for accurate measurements, and it is also time-consuming and complicated.

In order to solve the above problems, there have been several attempts to apply real time quantitative PCR to the first and third methods (Klein, D. et al., Gene Therapy 458-463, 2000; Sanburn, N and Cornetta, K., Gene Therapy 1340-1345, 1999). Such attempts have so far dealt with only the possibility of measuring retroviral titer by way of conducting real time quantitative PCR using a primer specific to EGFP (enhanced green fluorescence protein) or Neo gene used as the marker gene. However, in order to use real time quantitative PCR for measuring retroviral titer, it is required to develop primers specific only for virus genome, without interacting with human genome used as a target cell, and also to establish suitable PCR reaction conditions for such primers.

Recently, to prevent the generation of replication-competent retrovirus (RCR) in a packaging cell line through recombination, retrovirus vectors have been constructed by removing most retrovirus-originated sequences with the exception of the minimum sequence required for functioning as a vector. Therefore, when various types of retrovirus containing such retrovirus vectors are to be used, primers specific for the retrovirus-conserved sequences must be developed. Otherwise, it is not possible to accurately measure viral titer because of non-specific interactions with the genomic DNA or RNA of a host cell.

Further, the recent development of an equipment (ABI7700, PE Biosystems, Forster City, USA) for performing real time quantitative PCR makes it possible to quantitatively analyze the amount of template DNA. However, the analysis of viral titer with this quantitative equipment requires the development of retrovirus-specific primers.

The present inventors have therefore endeavored to meet the above need, and developed retrovirus-specific oligonucleotides that can be used for quantitatively analyzing retrovirus genomic DNA introduced into a host cell genome or retrovirus genomic RNA in a retrovirus containing solution. The retrovirus-specific oligonucleotides of the present invention can be effectively used for the rapid and quantitative measurement of retroviral titer.

### SUMMARY OF THE INVENTION

Accordingly, an object of the present invention is to provide retrovirus-specific oligonucleotides that can be used as primers for measuring retrovirus genomic DNA or RNA in a quantitative and rapid manner.

It is another object of the present invention to provide a method for rapidly quantifying retroviral titer by using the retrovirus-specific oligonucleotides as primers.

In accordance with one aspect of the present invention, there is provided retrovirus-specific oligonucleotides having the nucleotide sequences of SEQ ID Nos: 6, 11, 16 and 17.

In accordance with still another aspect of the present invention, there is provided a method for measuring retroviral titer by determining the amount of retrovirus genomic DNA or RNA using the retrovirus-specific oligonucleotides as primers for real time quantitative PCR.

Since the retrovirus-specific oligonucleotides of the present invention interact not with human genomic DNA but only with retrovirus genomic DNA or RNA, they can be used as primers for detecting various kinds of retrovirus.

### BRIEF DESCRIPTION OF THE DRAWINGS

The above and other objects and features of the present invention will become apparent from the following description of the invention, when taken in conjunction with the accompanying drawings which respectively show;

FIG. 1: the nucleotide sequence adjacent to retrovirus 3′ LTR region (GenBank #J02255; SEQ ID No: 1) and the recognition sites of the inventive oligonucleotides specific for the above nucleotide sequence;

FIG. 2: the nucleotide sequence of the packaging signal present in murine leukemia virus (MLV) genome (GenBank #J02255; 451-600) and the recognition sites of the inventive oligonucleotides specific for the above nucleotide sequence;

FIG. 3: the result of quantifying the retrovirus genomic DNA introduced into a host cell by real time quantitative PCR using the inventive oligonucleotide primer pair of SEQ ID Nos: 3 and 11; and

FIG. 4: the result of quantifying the retrovirus genomic RNA present in a virus containing solution by real time quantitative PCR using the inventive oligonucleotide primer pair of SEQ ID Nos: 16 and 17.

### DETAILED DESCRIPTION OF THE INVENTION

As used herein, the term “retrovirus-specific primers” means oligonucleotides that specifically interact not with human genomic DNA but only with retrovirus genomic DNA or RNA, and therefore, can be effectively used as PCR primers for selectively amplifying only the retrovirus genome when host cell genome containing retrovirus is hybridized.

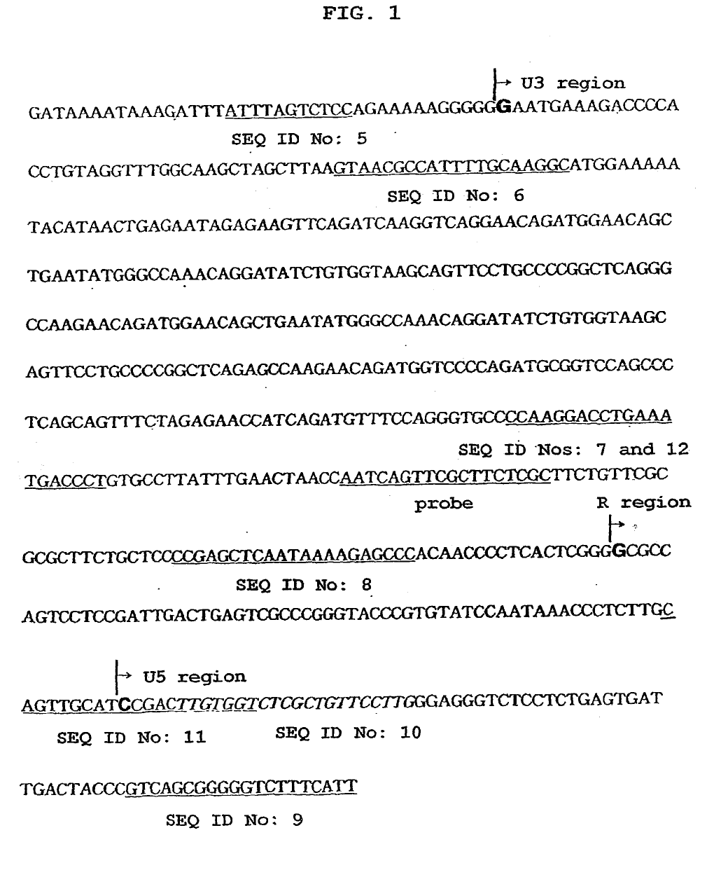

The oligonucleotide of SEQ ID No: 6 of the present invention specifically recognizes the 81-100^{th} nucleotide portion of the upstream U3 region in the nucleotide sequence adjacent to retrovirus 3′ LTR of SEQ ID No: 1 (GenBank #J02255; Shinnick, T. M. et al., Nature 293:543-548, 1981), and the oligonucleotide of SEQ ID No: 11, the 550-570^{th} nucleotide portion of the boundary between R and U5 regions (see FIG. 1).

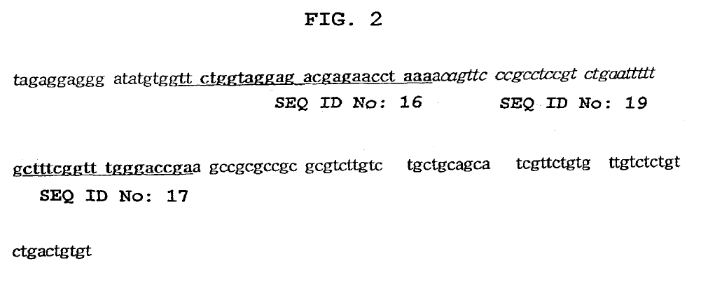

In addition, the oligonucleotides of SEQ ID Nos: 16 and 17 specifically recognize the 19-43^{rd} nucleotide portion and the 72-89^{th} nucleotide portion in the MLV packaging signal identified as SEQ ID No: 2, respectively, which is a conserved sequence in all MLV RNA (see FIG. 2).

Preferably, the method for measuring retroviral titer using the inventive oligonucleotides is performed by employing, but are not limited to, RT-PCR (reverse transcriptase polymerase chain reaction), semi-quantitative RT-PCR, QC-PCR (quantitative competitive PCR) or real time quantitative PCR, and more preferably, real time quantitative PCR which can measure the amount of PCR products generated during the amplification reaction in real time.

In the preferable embodiment, the inventive method for measuring retroviral titer can quantify retrovirus genomic DNA introduced into a host cell genome by using the inventive oligonucleotides as primers for real time quantitative PCR. In addition, it is possible to quantify retrovirus genome in a virus containing solution by synthesizing DNA from retrovirus genomic RNA via a reverse transcriptase reaction and then conducting real time quantitative PCR using the synthesized DNA as a template and the inventive oligonucleotides as primers.

The method for quantifying retrovirus genomic DNA by real time quantitative PCR using the inventive oligonucleotides comprises the steps of

1) infecting target cells with retrovirus;

2) purifying genomic DNA from virus-infected target cells;

3) conducting real time quantitative PCR using the purified genomic DNA as a template and the inventive oligonucleotides as primers; and

4) determining the retroviral titer by measuring the amount of virus genomic DNA in a unit of target cell genomic DNA

In addition, the method for quantifying retrovirus genomic RNA by real time quantitative PCR using the inventive oligonucleotides comprises the steps of:

1) purifying virus genomic RNA from a virus containing solution;

2) synthesizing DNA from the purified genomic RNA by a reverse transcriptase reaction;

3) conducting real time quantitative PCR using the synthesized DNA as a template and the inventive oligonucleotides as primers; and

4) determining the relative amount of virus genomic RNA in the virus containing solution used as a PCR template by quantifying the amplified PCR product.

The method for measuring retroviral titer by way of using the inventive oligonucleotides makes it possible to rapidly quantify the amount of retrovirus genomic DNA introduced into a target cell, or the amount of retrovirus genomic RNA in a retrovirus culture solution. Therefore, the inventive method can be effectively used for measuring the concentration of retrovirus vector used for gene therapy, detecting the presence of RCR in a retrovirus containing composition used for gene therapy, or detecting wild-type retrovirus.

Basically, the inventive method for detecting RCR can be applied to the BAG mobilization method (Pear, W. S. et al, Proc. Natl. Acad. Sci. USA 8392-8396, 1993). Briefly, this method comprises the steps of infecting with a solution of retrovirus vector to be tested a host cell in which wild-type retrovirus can replicate; conducting several subculture cycles; performing PCR using the cell culture solution as a template and the inventive oligonucleotides as primers; and detecting the retrovirus genomic RNA As mentioned above, since a retrovirus vector used for gene therapy lacks most of the viral gene coding sequences, it cannot grow in a host cell line in which wild-type retrovirus can replicate. Therefore, the retrovirus detected by this method can be regarded as RCR. Further, the inventive method can be used for detecting wild-type retrovirus from a patient who received gene therapy or a blood sample obtained of animal.

Hereinafter, the inventive method for measuring retroviral titer by way of using the inventive retrovirus-specific oligonucleotides as primers for real time quantitative PCR is described in detail.

1. Real time quantitative PCR

Real time quantitative PCR employs a double dye-labeled probe to detect the amplified PCR product in real time and can be performed according to the method and principle described by Heid (Heid, C. A. et al., Genome Res. 986-994, 1996).

The probe binds to a fragment to be amplified undergoes modification during the extension phase of the amplification procedure, and generates a detectable signal which indirectly represents the amount of amplified PCR product This method can measure the formation of PCR product in real time by detecting the signal. The probe used in this method can be hybridized to the 5′ and 3′ PCR primer binding site of a template DNA to be amplified The hybridized probe undergoes structural or conformational modification by the degrading action of DNA polymerase having 5→3′ nuclease activity during the extension phase. This modification is accompanied by shift in the absorbance, generating a detectable signal.

The probe comprises two labels, one being a reporter dye (R) labeled at 5′ end and the other, a quencher dye (Q) labeled at 3′ end. The reporter dye that can be employed in the present invention may be 6-carboxyfluorescein (FAM), 2′,4′,5′,7′-tetrachloro-4-7-dichlorofluorescein (TET), 2′,7′-dimethoxy-4′,5′-6-carboxyrhodamine (JOE) or VIC™ (PE Biosystems, USA), while the quencher dye may be 6-carboxytetramethyl rhodamine (TAMRA) or ([4-dimethylamine]azobenzene sulfonic acid (DABSYL).

In a preferred embodiment of using real time quantitative PCR, the present invention uses a probe labeled with FAM as the reporter dye and TAMRA as the quencher dye, which has the nucleotide sequence of FAM-SEQ ID No: 13-TAMRA having a region complementary to the retrovirus 3′ LTR region; or a probe labeled with VIC™ as the reporter dye and TAMRA as the quenched dye, which has the nucleotide sequence of VIC™-SEQ ID No: 19-TAMRA having a region complementary to the retrovirus packaging signal.

If the above two groups of compounds coexist in a solution, they undergo hybridization with each other to form a hairpin loop and come into contact with each other close enough to quench the emission of the reporter dye by the action of the quencher dye. Namely, the quencher dye acts as a quencher of the reporter dye.

However, when the probe is hybridized with a target gene fragment used as a PCR template, it exists in a linear form. As DNA polymerase performs the extension of the upstream primer by polymerization and comes into contact with the hybridized probe, the quencher dye labeled at the 5′ end of probe is degraded by the 5′ exonuclease activity of DNA polymerase and loses its activity to quench the reporter dye, resulting in an increase in fluorescence. Therefore, the fluorescence intensity of the reaction solution is proportional to the amount of target PCR product in the reaction solution.

Here, Ct (Threshold cycle) value means the number of reaction needed for the fluorescence to reach a threshold value, the threshold value being 10 times the standard deviation of the average values of baseline emission observed between reaction numbers 1 to 15.

Unlike the previous method for quantifying PCR product, the real time quantitative PCR based on the above principle has a merit in that it can scan the regime of reaction number which, in theory, occurs exponentially, and quantify the reaction number and the amount of template DNA via the correlation of 2^{ΔCt} (Heid, C. A et al., Genome Res. 986-994, 1996).

2. Method for measuring the titer of retrovirus introduced into a host cell genome

Needed in order to measure the retroviral titer by using real time quantitative PCR is a target cell line having one copy of retrovirus introduced in its genome. First, a packaging cell is infected with retrovirus to prepare a cell-free virus culture solution, and then, host cells are transduced with the cell-free virus culture solution at a multiplicity of infection (MOI) of below 0.1, preferably 0.5. After 24 hours, a single cell clone is formed on a plate using a limited dilution method. The clone infected with retrovirus is selected, cultured and its genomic DNA is purified. Southern blot analysis is conducted to examine whether or not the target cell is infected with one copy of the retrovirus. This selected cell line is chosen as 100% infected cell and used as a control.

The genomic DNA of 100% infected cell is mixed with that of normal host cell in various proportions to prepare standards which represent various levels of infection, and the infection level of a host cell is measured using the standards. Specifically, the genomic DNAs of host cell carrying one copy of retrovirus and the normal host cell are each isolated and subjected to restriction enzyme digestion to quantify the amount. These two kinds of DNAs are mixed in various proportions to prepare genomic DNA standards that represent infection levels of 100, 50, 25, 10, 5, 1, 0.5 and 0%, respectively. The genomic DNA standards are then subjected to real tine quantitative PCR to obtain Ct values at various infection levels, and a standard curve is prepared by plotting the Ct values against infection levels.

The genomic DNA of the experimental group cell line to be tested is purified by the same method described above, and subjected to real time quantitative PCR to obtain a Ct value. It is then possible to determine the infection level of the target cell by comparing the Ct value thus obtained with the standard curve. This result can be used to calculate the gene delivery efficiency of the retrovirus and the viral titer.

The PCR reaction solution used consists of 5 μl of genomic DNA, 1 μl each of the primers having nucleotide sequences of SEQ ID Nos: 6 and 11, and 2 μl of double-dye labeled probe, and is adjusted to a final reaction volume of 25 μl with distilled water. The PCR is preferably conducted for 2 min at 50° C. and 10 min at 95° C., followed by 40 to 45 cycles of 15 sec at 95° C. and 1 min 30 sec at 60° C.

In a preferred embodiment, the present invention uses human HT1080 cell line having one copy of MT5-IDUA retrovirus introduced therein. MT5-IDUA retrovirus is prepared by inserting human iduronidase gene into MT5 retrovirus vector which has been applied for a patent by the present inventors (Korea Patent Publication Laid-Open No: 1998-24847; KCCM-10205). It has been confirmed that as the infection level increases, the Ct value decreases, and that there exists an excellent correlation (r²=0.998) between the infection level and the Ct value.

3. Method for measuring retroviral titer in a retrovirus containing solution

The method for measuring retrovirus genomic DNA introduced in a target cell has a merit in that it can detect the actually infecting virus, but it is cumbersome to go through the transduction procedure. In order to simplify such method for measuring viral titer, there has been developed a method tnat measures the total retrovirus genomic RNA in a solution. In this method, oligonucleotides having the nucleotide sequences of SEQ ID Nos: 16 and 17 can be employed as primers for real time quantitative PCR.

First, a retrovirus containing solution or retrovirus genomic RNA purified therefrom is subjected to a reverse transcriptase reaction to synthesize DNA. In case of using a virus containing solution, retrovirus genomic RNA dissolves into the solution during high temperature denaturation procedure at the early stage of the reverse transcriptase reaction, making it possible to omit a separate RNA extraction step, which results in increased experimental efficiency and enhanced process economy. Further, retrovirus genomic RNA can be extracted from the cell culture solution containing retrovirus or other solution using any of the methods known in the art and subjected to reverse transcription.

cDNA synthesized by reverse transcription is employed as a template in conducting the real time quantitative PCR according to the above-mentioned method using the oligonucletodies having nucleotide sequences of SEQ ID Nos: 16 and 17 as primers, wherein the oligonucleotides specifically recognize the packaging signal region of MLV genome.

In another embodiment of the present invention, the viral titer of an MT5-IDUA retrovirus containing solution is measured by the inventive method. The observed Ct values show an excellent correlation (r32 0.9993) with the amount of the retrovirus in the solution, which confirms that it is possible to measure the relative amount of cDNA from the Ct value calculated by real time quantitative PCR.

The following Examples and Test Examples are given for the purpose of illustration only, and are not intended to limit the scope of the invention.

### EXAMPLE

### Example 1

Preparation of human cell line infected with one copy of retrovirus

(1-1) Preparation of MT5-IDUA retrovirus vector

In order to design primers that can detect various kinds of retrovirus, it is preferable to use a retrovirus vector which has sequences common to all kinds of retrovirus. MT5 vector developed by the present inventors (Korea Patent Publication Laid-Open No: 1998-24847; KCCM-10205) has been prepared by removing all other retrovirus-originated sequences but the minimum sequences needed for acting as a gene delivery vehicle. In the present invention, MT5-IDUA vector was prepared by inserting human iduronidase gene into MT5 vector.

First, RNA was extracted from human foreskin fibroblast (HFF) by the Tryzol (Gibco BRL, USA) method, and subjected to a reverse transcriptase reaction to synthesize cDNA. DNA fragments containing human iduronidase gene were amplified using the synthesized cDNA as a PCR template and the synthetic oligonucleotides of SEQ ID Nos: 3 and 4 as primers (which recognize the 76-96^{th} and 2041-2061^{st} nucleotide portions of human iduronidase gene [GenBank #M74715], respectively). The PCR reaction solution was prepared by mixing 1 μl of template DNA, 1 μl of 10 pmol/μl each primer, 10 μl of 10 mM dNTP, 3.5 unit of Expand High Fidelity enzyme (Gibco BRL, USA) and 10 μl of enzyme buffer solution and adjusted to a final volume of 100 μl. The PCR condition was 30 cycles of 1 min at 94° C., 1 min at 55° C. and 1 min 30 sec at 72° C. As a result, about 2,000 bp of the PCR product was amplified and cloned into pGEM T easy vector (Promega, Wis., USA) to obtain pGEM T easy-IDUA vector. pGEM T easy-IDUA vector was then subjected to BamHI restriction enzyme digestion to obtain 1,986 bp of the human iduronidase gene fragment, and this fragment was inserted at the BamHI restriction enzyme site of MT5 (KCCM-10205) to obtain MT5-IDUA vector.

(1-2) Preparation of human cell line infected with one copy of retrovirus

In order to prepare a cell line infected with one copy of retrovirus, the retrovirus containing MT5-IDUA vector was prepared as follows.

First, MT5-IDUA vector DNA prepared in Example (1-1) was transfected into 293T cells Yu, S. S. et al., Gene Therapy 7:797-804, 2000) together with pVM-gp plasmid DNA (TAKARA SHUZO, Japan) expressing gag-pol gene of murine leukemia virus (MLV) and pRV67 plasmid DNA (Mitrophanous, K. A. et al., Gene Therapy 6:1808-1818, 1999) expressing VSV-G (Vesicular Stomatitis Virus-G) protein, and transferred into a new medium after 8 hours. The transfected cells were further cultured for 48 hours and filtered through a 0.45 μm filter paper to obtain cell-free virus.

HT1080 cell line (ATCC #CCL-121) was traduced with the MT5-IDUA cell-free retrovirus thus obtained at an MOI (multiplicity of infection) of below 0.05. After 24 hours, the target cells were diluted to a concentration of 0.3 cells/well on a 96 well plate to obtain a single clone, and the single clone carrying introduced retrovirus was selectively cultured by treating wit G418 (1 μg/μl, Gibco BRL, USA) for 14 days. The genomic DNA was purified from the selected clone and subjected to southern blot analysis using the probe specific for the nucleotide sequence of packaging signal. As a result, it has been confirmed that one copy of retrovirus is introduced into the genome of the target cell.

The selected clone having one copy of retrovirus vector MT5-IDUA introduced to its genome is designated HT1080:MT5-IDUA.

### Example 2

Development of specific primers for retrovirus genomic DNA introduced into host cell

In order to develop primers specific only for the retrovirus genomic DNA which do not interact with human genomic DNA, various types of oligonucleotides have been designed and subjected to PCR using the genomic DNAs obtained from HT1080:MT5-IDUA cell line prepared in Example 1 and wild-type HT1080 cell line.

To extract the total genomic DNA from HT1080 cell line, HT1080 cell precipitate was added to a TES (10 mM Tris-HCl; 1 mM EDTA; 0.7% SDS) buffer solution and treated with 400 g/ml of proteinase K for 2 hours at 50° C. The total genomic DNA was then purified from HT1080 cell line via phenol/chloroform extraction and ethanol precipitation. The purified genomic DNA was quantified by treating with restriction enzyme EcoRI, and 1 μg of genomic DNA was subjected to PCR. The PCR reaction solution was prepared by mixing 1 μl of genomic DNA, 1 μ; each of the primers (200 nM), and 5 μl of an enzyme buffer solution and adjusted to a final volume of 50 μl. The PCR condition was 30 cycles of 1 min at 94° C., 1 min at 60° C. and 1 min 30 sec at 72° C.

The result of examining the specificity of several primer pairs of oligonucleotides designed by the present inventors under the above PCR conditions shows that the primer pair of SEQ ID Nos: 6 and 11 are specific to the retrovirus genomic DNA and do not interact with human genomic DNA (FIG. 1). Table 1 lists the PCR results obtained with various oligonucleotide primers.

TABLE 1

| | | Result | | |
|---|---|---|---|---|
| Combination of primer pair | | Viral | Human | |
| Forward | Reverse | genome | genome | Estimation |
| SEQ. ID No: 5 | SEQ. ID No: 9 | − | + | Unsuitable |
| SEQ. ID No: 5 | SEQ. ID No: 11 | − | + | Unsuitable |
| SEQ. ID No: 5 | SEQ. ID No: 12 | − | + | Unsuitable |
| SEQ. ID No: 6 | SEQ. ID No: 9 | + | + | Unsuitable |
| SEQ. ID No: 6 | SEQ. ID No: 10 | + | + | Unsuitable |
| SEQ. ID No: 6 | SEQ. ID No: 11 | + | − | Suitable |
| SEQ. ID No: 6 | SEQ. ID No: 12 | + | + | Unsuitable |
| SEQ. ID No: 7 | SEQ. ID No: 9 | + | + | Unsuitable |
| SEQ. ID No: 7 | SEQ. ID No: 10 | + | + | Unsuitable |
| SEQ. ID No: 7 | SEQ. ID No: 11 | + | + | Unsuitable |
| SEQ. ID No: 8 | SEQ. ID No: 9 | + | + | Unsuitable |
| SEQ. ID No: 8 | SEQ. ID No: 10 | + | + | Unsuitable |
| SEQ. ID No: 8 | SEQ. ID No: 11 | + | + | Unsuitable |

+reaction occurred,
−reaction not occurred

As illustrated in Table 1, the inventive primer pair of SEQ ID Nos: 6 and 11 specifically recognizes the conserved LTR sequence of the retrovirus genomic DNA introduced into the host cell genome, and therefore, it can be effectively used for detecting all retrovirus having the LTR structure.

### Example 3

Quantification of retrovirus genomic DNA using real time quantitative PCR

To examine whether the retrovirus genomic DNA introduced into the host cell can be quantified by applying the primer pair developed in Example 2 to real time quantitative PCR, the following experiment was performed.

First, the genomic DNAs were purified from HT1080:MT5-IDUA cell line obtained from Example (1-2) and wild-type HT1080 cell line, respectively, and quantified by treating with restriction enzyme EcoRI. The purified DNAs were then mixed together in various proportions to obtain genomic DNA standards having infection levels of 100, 50, 25, 10, 5, 1, 0.5, 0.1 and 0%, respectively, followed by examining whether real time quantitative PCR reflects the infection levels of standard genomic DNAs or not.

Real time quantitative PCR was performed according to the TaqMan™ analyzing method (Heid C. A. et al., Genome Res. 986-994, 1996) with ABI17700 PCR equipment (PE Biosystems, Foster City, US). Used together with the Inventive primer pair of Example 2 was the probe having the nucleotide sequence of FAM-SEQ ID No: 13-TAMRA which is complementary to retrovirus LTR region, the probe being doubly labeled at its 5′ and 3′ ends with 6-carboxyfluorecein (FAM) as the fluorescence reporter dye (R) and 6-carboxytetramethyl rhodamine (TAMRA) as the quencher dye, respectively.

The PCR reaction solution was prepared by mixing 5 μl of genomic DNA, 25 μl of TaqMan™ buffer solution (2×), 1 μl of probe (2 μM, 1 μl each of the primers (200 nM) and 17 μl of distilled water and adjusted to a final volume of 50 μl. The PCR condition consisted of 45 cycles of 15 sec at 95° C. and 1 min 30 sec at 60° C. after the initial denaturation for 2 min at 50° C. and 10 min at 95° C. To confirm the amount of genomic DNA used, PCR was performed using the primer pair of SEQ ID Nos: 14 and 15 which specifically recognize human β-actin gene and the probe having the nucleotide sequence of VIC™-SEQ ID No. 18-TAMRA specific for β-actin gene, as a control.

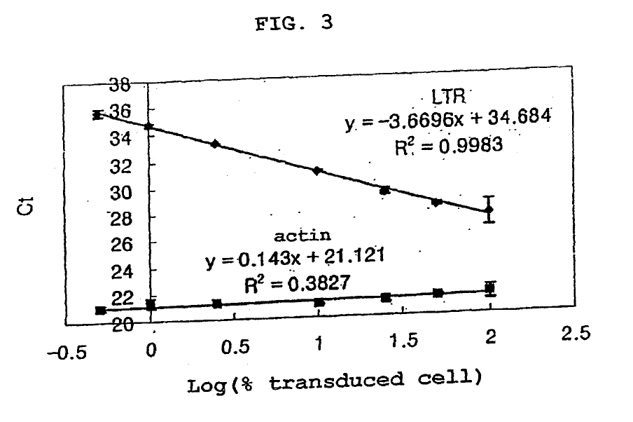

The result showed that as the infection level increased in the order of 0, 0.1, 0.5, 1, 5, 10, 25, 50 and 100%, the Ct (Threshold cycle) value which represents the reaction number for the fluorescence to reach the threshold value, decreased proportionately. This result indicates that there exists a significant correlation (r²=0.998) between the infection level and Ct value (FIG. 3). Therefore, the inventive primer pair developed in Example 2 can be effectively used to detect retrovirus genomic DNA using real time quantitative PCR.

### Example 4

Comparison of the method for measuring the retroviral titer using real time quantitative PCR with the method for measuring the retroviral titer using MDR selective marker gene

(4-1) Preparation of PG13 cell line producing MTM-GC retrovirus

To compare the method for measuring the retroviral titer using real time quantitative PCR performed in Example 3 with the previous method for measuring the retroviral titer using MDR (multi-drug resistance) selective marker gene, the cell line producing retrovirus which expresses MDR gene was prepared as follows.

Human glucocerbrosidase gene was inserted into the multi-cloning site of MTM vector (Korea Patent Publication Laid-Open No: 1998-24847) to obtain MTM-GC retrovirus vector, MTM vector containing the nucleotide sequence of MDR gene fused with IRES (internal ribosomal entry site) downstream of the multi-cloning site and being capable of expressing the target gene with MDR gene.

To prepare PG13 cell line that produces MTM-GC retrovirus, MTM-GC vector DNA was transfected into 293T cells together with pVM-gp plasmid DNA and pRV67 plasmid DNA according to the method described in Example (1-2), and the cell culture solution was filtered to obtain cell-free virus.

PG13 cell line (ATCC CRL10686, MD, USA) was transduced with the MTM-GC cell-free retrovirus obtained above. After 24 hours, cells were subjected to a limited dilution method on a 96 well plate to obtain a clone of a single cell type. The clone having introduced retrovirus was selected by treating with vincristine (25 ng/ml) for 14 days.

One of the selected cell line clones showing a high viral titer has been chosen and designed PG13:MTM-GC cell line.

(4-2) Comparison of the method using real-time quantitative PCR with using MDR selective marker gene for measuring retroviral titer

Cell-free virus was obtained from PG13:MTM-GC cell line prepared in Example (4-1), and transduced into HT1080 target cells at different concentrations. After 24 hours, the transduced target cells were divided into 3 groups (Group I, II and III) and incubated. The efficiency of the method using real time quantitative PCR for measuring the retroviral titer was compared with that of the method using MDR selective marker gene by conducting the following experiments with these 3 groups of trasnduced target cells.

After Group I was further cultured for 48 hours, its genomic DNA was purified and the infection level of the target cell with MTM-GC retrovirus was examined using real time quantitative PCR according to the method described in Example 3. Group II was further cultured for 24 hours and examined what % of the target cell expresses MDR gene by FACS analysis (Fluorescence activated cell sorter analysis; Aran, J. M. et al., Cancer Gene Therapy 195-206, 1998) using an antibody specific for MDR protein (KAMIYA BIOMEDICAL COMPANY, USA). After the target cells of Group III were diluted-by 1/100, 1/500, 1/1,000and 1/5,000 and treated with vincristine, the number of colonies infected with MTM-GC retrovirus was counted. These results are shown in

TABLE 2

| Virus | Group I | | Group II | | |
|---|---|---|---|---|---|
| containing | | Estimated | | Estimated titer | Group III |
| solution | % | titer (×10⁵) | % | (×10⁵) | (×10⁵) |
| A | 100.5 ± 21.8 | >5.0 | 65.7 ± 2.9 | 5.4 | 2.9 ± 1.2 |
| B | 7.8 ± 4.8 | 0.4 | 22.4 ± 3.6 | 1.3 | 0.7 ± 0.4 |

Based on the result, it has been confirmed that there is a good correlation between the real time quantitative PCR method using the inventive oligonucleotide primers and the vincristine-resistance colony counting FACS method.

### Example 5

Method for measuring retrovirus genomic RNA using real time quantitative PCR

The inventive method described in Examples 3 and 4 measures the retrovirus genomic DNA introduced into a target cell genome. This method has the advantage of being able to detect the retrovirus actually infecting the target cell, but is cumbersome because a transduction step is needed. To solve this problem, there has been developed a method for measuring the total retrovirus genomic RNA in a solution, not retrovirus genomic DNA This method has a disadvantage in that it measures not the infectious retrovirus RNA but the total retrovirus RNA, but is simpler than the method for detecting the retrovirus genomic DNA.

Specifically, cDNA was synthesized by RT-PCR using a one-step reverse transcriptase amplification kit (TaqMan Gold One-step RT-PCR kit, PE Applied Biosystems, USA) which employs 1, 0.1, 0.01, 0.001 and 0.0001 μl of a solution containing 1×10⁶ virus/ml of MT5-IDUA retrovirus vector as a template. RT-PCR was performed using the virus containing solution directly without a separate RNA extraction step. This was possible because the retrovirus was denatured at a high temperature denaturation step during the early stage of RT-PCR, leaking the retrovirus genomic RNA into the solution.

In order to amplify the retrovirus genome sequence in the retrovirus containing solution at a various concentration, PCR was performed by using the synthesized cDNA as a template, the probe having the nucleotide sequence of VIC™-SEQ ID No: 19-TAMRA, and the synthetic oligonucleotides of SEQ ID Nos: 16 and 17 as primers. The one step RT-PCR, the successive reverse transcriptase and amplification reactions carried out in same tube, was performed using ABI7700 PCR equipment The RT-PCR solutions was prepared by mixing 1 μl of retrovirus solution, 12.5 μl of TaqMan™ master mix buffer solution (2×), 2 μl of probe (2 μM), 2 μl each of the primers (10 pmol/μl), 0.5 μl of 40×MultiScribe and RNase inhibitor mix, 5 μl of distilled water to a final volume of 25 μl. The PCR condition consisted of 40 cycles of 15 sec at 95° C. and 1 min at 60° C.

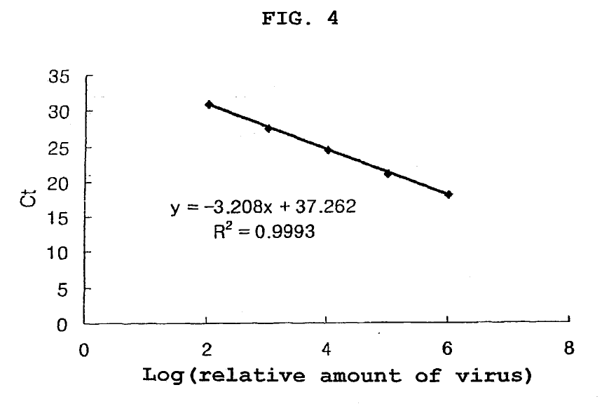

As a result, it was found that as the relative viral titer of the retrovirus containing solution increases, Ct value, which represents the reaction number for the fluorescence to reach the threshold value, decreases proportionately, and that there exists a good correlation (r²=0.9993) between the amount of retrovirus in the solution and Ct value (FIG. 4). In FIG. 4, the log[relative amount of virus] value of the X axis is obtained by setting the value for 1×10⁶ virus/ml retrovirus solution as a standard, measuring the fluorescence of the samples containing 1, 0.1, 0.01, 0.001 and 0.0001 μl of the standard solution, and regarding the measured fluorescence as the-relative amount of virus. Ct value thus obtained reflects the relative amount of template cDNA. This result suggests that the inventive primer pair of SEQ ID Nos: 16 and 17 can be effectively applied for detecting retrovirus genomic RNA using real timid quantitative PCR.

While the embodiments, of the subject invention have been described and illustrated, it is obvious that various changes and modifications can be made therein without departing from the spirit of the present invention which should be limited only by the scope of the appended claims.

What is claimed:

1. A quantitative method for measuring retrovirus genomic DNA or RNA by using retrovirus-specific oligonucleotides as primers for polymerase chain reaction (PCR).

2. The method of claim 1, wherein the retrovirus-specific oligonucleotides are the nucleotides of SEQ ID Nos: 6 and 11 which specifically recognize a portion of the long terminal repeat (LTR) of retrovirus genome.

3. The method of claim 1, wherein the retrovirus-specific oligonucleotides are the nucleotides of SEQ ID Nos: 16 and 17 which specifically recognize a portion of the packaging signal of murine leukemia retrovirus (MLV) genome.

4. The method of claim 1, wherein the PCR is selected from the group consisting of RT-PCR (reverse transcriptase polymerase chain reaction), semi-quantitative RT-PCR, QC-PCR (quantitative competitive PCR) and real time quantitative PCR.

5. The method of claim 4, which comprises the steps of:
1) infecting target cells with retrovirus;
2) purifying genomic DNA from virus-infected target cells;
3) conducting real time quantitative PCR using the purified genomic DNA as a template and the oligonucleotides of SEQ ID Nos. 6 and 11 as primers; and
4) determining the retroviral titer by measuring the amount of viral genomic DNA in a unit of target cell genomic DNA.

6. The method of claim 4, which comprises the steps of:
1) purifying virus genomic RNA from a virus culture solution or a virus containing solution;
2) synthesizing DNA from the genomic RNA using reverse transcriptase;
3) conducting real time quantitative PCR using the synthesized DNA as a template and the oligonucleotides of SEQ ID Nos: 16 and 17 as primers; and
4) determining the relative amount of virus genomic RNA in the sample solution by quantifying the amplified PCR product.

7. The method of claim 5 or 6, which further uses in Step (3) a probe labeled with a reporter dye (R) at its 5′ end and a quencher dye (Q) at its 3′ end.

8. The method of claim 7, wherein the reporter dye is selected from the group consisting of 6-carboxyfluorescein (FAM), 2′,4′,5′,7′-tetrachloro-4-7-dichlorofluorescein (TET), 2′,7′-dimethoxy-4′,5′-6-carboxyrhodamine (JOE) and VIC™, and the quencher dye is selected from the group consisting of 6-carboxytetramethyl rhodamine (TAMRA) and [4-dimethylamime]azobenzene sulfonic acid (DABSYL).

9. An oligonucleotide having the nucleotide sequence of SEQ ID No: 6 which specifically recognizes the portion of LTR (long terminal repeat) conserved in retrovirus genome.

10. An oligonucleotide having the nucleotide sequence of SEQ ID No: 11 which specifically recognizes the portion of LTR conserved in retrovirus genome.

11. An oligonucleotide having the nucleotide sequence of SEQ ID No: 16 which specifically recognizes the portion of packaging signal conserved in murine leukemia virus genome.

12. An oligonucleotide having the nucleotide sequence of SEQ ID No: 17 which specifically recognizes the portion of packaging signal conserved in murine leukemia virus genome.


SEQUENCE LISTING (19 sequences)

SEQ ID NO: 1 - DNA, 634 bp, Retrovirus 3′ LTR
gataaaataaagatttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagagccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 2 - DNA, 150 bp, packaging signal of Murine Leukemia Virus
tagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgt

SEQ ID NO: 3 - DNA, 33 bp, Artificial Sequence
  specific forward primer for iduronidase
ggatccgatatcagcacgcgtggccatgcgtcc

SEQ ID NO: 4 - DNA, 33 bp, Artificial Sequence
  specific reverse primer for iduronidase
ggatccagatcttcagcacaggctcatggattg

SEQ ID NO: 5 - DNA, 20 bp, Artificial Sequence
  oligonucleotide
aaaggatccatttagtctcc

SEQ ID NO: 6 - DNA, 20 bp, Artificial Sequence
  oligonucleotide
gtaacgccattttgcaaggc

SEQ ID NO: 7 - DNA, 21 bp, Artificial Sequence
  oligonucleotide
ccaaggacctgaaatgaccct

SEQ ID NO: 8 - DNA, 21 bp, Artificial Sequence
  oligonucleotide
ccgagctcaataaaagagccc

SEQ ID NO: 9 - DNA, 26 bp, Artificial Sequence
  oligonucleotide
gaattcaatgaaagacccccgctgac

SEQ ID NO: 10 - DNA, 21 bp, Artificial Sequence
  oligonucleotide
caaggaacagcgagaccacaa

SEQ ID NO: 11 - DNA, 21 bp, Artificial Sequence
  oligonucleotide
accacaagtcggatgcaactg

SEQ ID NO: 12 - DNA, 21 bp, Artificial Sequence
  oligonucleotide
agggtcatttcaggtccttgg

SEQ ID NO: 13 - DNA, 28 bp, Artificial Sequence
  double dye-labeled probe
aatcagttcgcttctcgcttctgttcgc

SEQ ID NO: 14 - DNA, 25 bp, Artificial Sequence
  forward primer for human beta-actin
tcacccacactgtgcccatctacga

SEQ ID NO: 15 - DNA, 25 bp, Artificial Sequence
  reverse primer for human beta-actin
cagcggaaccgctcattgccaatgg

SEQ ID NO: 16 - DNA, 25 bp, Artificial Sequence
  oligonucleotide
ttctggtaggagacgagaacctaaa

SEQ ID NO: 17 - DNA, 18 bp, Artificial Sequence
  oligonucleotide
tcggtcccaaaccgaaag

SEQ ID NO: 18 - DNA, 26 bp, Artificial Sequence
  double dye-labeled probe for beta actin
atgccctcccccatgccatcctgcgt

SEQ ID NO: 19 - DNA, 26 bp, Artificial Sequence
  double dye-labeled probe
cagttcccgcctccgtctgaattttt